(19) United States
(12) Patent Application Publication
(10) Pub. No.: US 2005/0266073 A1
(43) Pub. Date: Dec. 1, 2005

(54) Fast disintegrating granules containing bromhexin/bromhexinhydrochloride, process for their preparation, and their use in veterinary medicine

(75) Inventors: Martin Folger, Ingelheim (DE); Stefan Henke, Kirchen (DE); Jens Schmalz, Hueffelsheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Vetmedica GmbH, Ingelheim (DE)

(21) Appl. No.: 11/127,971

(22) Filed: May 12, 2005

(30) Foreign Application Priority Data
May 12, 2004 (DE) 10 2004 023 930

(51) Int. Cl. A61K 9/20

(52) U.S. Cl. 424/464

(57) ABSTRACT

The invention relates to rapidly dissolving granules obtainable by preparing an aqueous granulating liquid containing at least one binder; spraying the granulating liquid obtained in step (a) in a fluidized bed by the counter-flow method at a constant air supply temperature on to a carrier containing at least one carbohydrate, wherein bromhexin/bromhexinhydrochloride and citric acid are used independently of one another in the granulating liquid and/or carrier. For the first time the teaching according to the invention provides a water soluble granulated preparation which contains bromhexin/bromhexinhydrochloride as active substance. These granules according to the invention are water soluble in spite of the fact that bromhexin/bromhexinhydrochloride per se is almost insoluble in water, i.e., the granules are fully dissolved in water within a few minutes and release the active substance in its entirety.

This application claims priority under 35 U.S.C. 119(a) of Germany Patent Application No. 10 2004 023 930 filed on May 12, 2004, which priority application is incorporated herein by reference in its entirety.

### BACKGROUND TO THE INVENTION

1. Field of the Invention

The invention relates to fast dissolving granules of bromhexin/bromhexin-hydrochloride, a process for preparing them and their use.

2. Description of the Prior Art

Bromhexin, like its hydrochloride, is a mucolytic active substance with the chemical name N-cyclohexyl-N-methyl-(2-amino-3,5-dibrombenzyl)amine and the chemical formula:

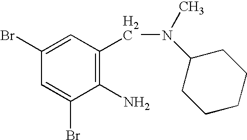

It is a cream colored crystalline solid which is soluble with difficulty in water, with a solubility of 1 g/250 ml. Bromhexinhydrochloride (Bisolvon®) is even more difficult to dissolve in water and dissolves with difficulty in ethanol, methanol and methylene chloride. It is therefore not surprising that known pharmaceutical formulations containing bromhexin(-hydrochloride) are largely used in formulations which are released over a longer period or after a time delay, resulting in delayed or sustained release. The release of the active substance is controlled by the dissolving or corresponding permeability of an applied film or by a deliberately selected porosity of a matrix in which the active substance is present in finely divided form. Pharmaceutical compositions containing bromhexin/bromhexinhydrochloride with a delayed release are known for example from U.S. Pat. No. 4,814,176 or U.S. Pat. No. 4,777,033.

Numerous combined preparations containing bromhexin(-hydrochloride) are also known, wherein two or more pharmaceutically active ingredients are present together in the formulation. Reference is made purely by way of example to U.S. Pat. No. 5,393,531 which discloses an aqueous solution or dispersion with at least two different pharmaceutically active substances, including bromhexin; WO 9633704, in which a composition of ibuprofen and bromhexinhydrochloride but in individual granules is described or JP 63060926, in which bromhexinhydrochloride is administered in conjunction with a fever-reducing and pain relieving agent.

The known Bisolvon®-powder is currently on the market for the treatment of chronic bronchopneumonia and enzootic pneumonia in pigs. Various preparations for human drugs are also commercially available. For veterinary use, in particular, it is obvious that sick animals can be successfully treated through their drinking water.

However, as Bisolvon®-powder is not soluble in drinking water in the necessary concentrations, other preparations, and methods of administration have hitherto been required. It would therefore be a major advantage to develop a suitably water soluble preparation.

There is already a proposal for this in the prior art:

Thus U.S. Pat. No. 5,837,285 describes tablets for oral administration to humans or animals, which dissolve rapidly in the mouth and contain a pharmaceutically active ingredient (drug) and a readily water soluble carrier of xylitol mixed with lactose or mannitol, while bromhexinhydrochloride is mentioned among a large number of possible drugs. In contrast to the conventional manufacture of tablets, after the wet mixture of drug and carrier has been kneaded, first of all the damp mixture is compressed into the form of tablets and the compressed tablets are then dried.

There is therefore a need to develop a specially tailored water soluble formulation for an active substance such as bromhexin or the hydrochloride thereof.

The present invention therefore sets out to provide a preparation containing bromhexin or bromhexinhydrochloride which has sufficient solubility in water to allow the active substance to be released relatively fast and completely. An orally administered pharmaceutical formulation is to be prepared. Another aim of the present invention is to provide a method which allows the formulation to be produced in a reproducible manner at a consistently high quality and without excessive expenditure.

### BRIEF DESCRIPTION OF THE FIGURE

FIG. 1 shows an embodiment of the process according to the invention.

### DETAILED DESCRIPTION OF THE INVENTION

The objective described above is achieved by means of the features of claim 1. According to this a fast dissolving granulated preparation is produced which can be prepared by
(a) preparing an aqueous granulating liquid containing at least one binder;
(b) spraying the granulating liquid obtained in step (a) in a fluidized bed in a counter-flow or counter-current process at a constant air supply temperature on to a substrate containing at least one carbohydrate,
   bromhexin/bromhexinhydrochloride and citric acid being used independently of one another in the granulating liquid and/or substrate.

In other words, the present invention provided a method of producing fast dissolving granules comprising: preparing an aqueous granulating liquid comprising at least one binder; preparing a carrier comprising at least one carbohydrate; and fluidizing the carrier; and spraying the granulating liquid onto the fluidized carrier to form granules, wherein bromhexin/bromhexinhydrochloride is present in the aqueous granulating liquid, or the carrier, or both the aqueous granulating liquid and the carrier, and wherein citric acid is present in the aqueous granulating liquid, or the carrier, or both the aqueous granulating liquid and the carrier independent of the bromhexin/bromhexinhydrochloride. A fast dissolving granule made according to the method of the present invention will at least comprise: bromhexin or bromhexin hydrochloride, citric acid, at least one binder, and at least one carbohydrate.

Thus, the teaching according to the invention for the first time provides a water soluble granulated preparation, particularly fast dissolving granules, which contain as active substance bromhexin/bromhexinhydrochloride which has very limited solubility in water. “Fast dissolving granules” according to the present invention indicates that the granules dissolve completely in water within a few minutes. In tests, a dissolving time of less than 15 minutes was achieved in a USP Paddle Apparatus (50 rpm) in water (drinking water, Ingelheim, normal pH and hardness) at ambient temperature, during which time the granules dissolve completely and all the active substance was released.

The term “bromhexin/bromhexinhydrochloride” for the purposes of the present invention denotes that either bromhexin or its hydrochloride is present in the fast dissolving granules. Thus, the granules according to the invention contain bromhexin or bromhexinhydrochloride as active substance which is added to the granulating liquid and/or to the substrate, most preferably to the substrate. Advantageously, the bromhexin/bromhexinhydrochloride is used in step (a) and/or (b) in an amount of approximately 0.5% (w/w) to approximately 2% (w/w), especially approximately 0.75% (w/w) to approximately 1.25% (w/w).

Moreover, a salt forming agent is present in the form of citric acid in the granulating liquid and/or carrier, most preferably in the carrier. If bromhexin/bromhexinhydrochloride is also present in the carrier, it has proved advantageous for the two components to be intimately physically mixed together. Most preferably the citric acid in step (a) and/or (b) is present in a range from approximately 25% (w/w) to approximately 95.5% (w/w), more particularly approximately 37.5% (w/w) to approximately 62.5% (w/w).

Within the scope of the invention the binder present in the granulating liquid is not particularly restricted. Any binder known in the pharmaceutical field may be used. Examples include polyvinylpyrrolidone, cellulose ether, gelatin, polyvinylacetates, acrylic resins, copolymers and mixtures thereof as well as starch and starch derivatives.

By “cellulose ethers” are meant according to the invention cellulose derivatives produced by partial or total substitution of the hydrogen atoms or hydroxy groups by alkyl and/or (ar)alkyl groups. These may also contain other functional groups. It is known that their solubility in water is dependent on the nature and number of ether groups introduced. Cellulose mixed ethers are also included.

Particularly preferred cellulose ethers are therefore methylcellulose, propylcellulose, hydroxyethylcellulose, hydroxypropylcellulose, methylhydroxyethylcellulose, methylhydroxypropylcellulose, hydroxypropylcellulose, hydroxypropylethylcellulose and hydroxypropylmethylcellulose. Hydroxypropylmethylcellulose and methylcellulose are particularly preferred.

Preferred polyvinylpyrrolidones are for example Kollidone® and PVP 25000.

Preferred acrylic resins include for example Eudragit®.

Most particularly preferred are cellulose ethers, optionally in admixture with polyvinylpyrrolidone, optionally together with an acrylic resin and a polyethylenglycol.

Expediently, the binder or binders in step (a) is or are present in an amount of from approximately 1% (w/w) to approximately 6% (w/w), particularly approximately 2% (w/w) to approximately 4% (w/w), based on dry product.

According to a particularly preferred embodiment the granulating liquid is produced in the form of a solution, i.e., the binder or binders are preferably dissolved in the granulating liquid, so that a granulating liquid solution is produced in step (a). However, a dispersed system may also be produced.

The carrier used in step (b) according to the invention contains one or more carbohydrates. These are not particularly restricted and can be selected from the mono-, oligo-, or polysaccharides and mixtures thereof. The mono- and oligosaccharides which may be used according to the invention and which are also categorized as “sugars” include for example monosaccharides such as glucose, mannose, galactose, fructose, sorbose, ribose, xylose and arabinose; disaccharides such saccharose, lactose, maltose, cellobiose and dextrin or maltodextrin. Mixtures of glucose and lactose are most particularly preferred.

Examples of polysaccharides which may be used in the carrier according to the invention include starch, starch derivatives, gum arabic or tragacanth.

Preferably, the carbohydrate or carbohydrates in step (b) are used in an amount of from approximately 35% (w/w) to approximately 75% (w/w), particularly approximately 40% (w/w) to approximately 60% (w/w).

In the present invention, the “starch” is subsumed under polysaccharides and hence under the carbohydrates. Admittedly, starch is also known to have binding qualities, but for reasons of clarity of classification for those skilled in the art this category is selected.

It may be advantageous if in addition to the binder one or more carbohydrates are added to the granulating liquid according to step (a). Most preferably, starch is additionally added to the granulating liquid.

Naturally, one or more excipients may be used in the granulating liquid and/or in the carrier, these excipients being selected from among the sweeteners, flavorings, sugar alcohols, fillers, disintegrants, break up agents, flow agents or flow regulators, lubricants, mould release agents, pH correctors, particularly buffers, antioxidants and colorings.

By “sweeteners” are meant according to the invention compounds of a synthetic or natural origin which have no calorific value or only negligible calorific value compared with their sweetening power. Particularly suitable sweeteners according to the invention are saccharine, aspartame, cyclamate and acesulfame and the salts thereof.

Other excipients may be sugar alcohols which are not sugars but are polyhydroxy compounds formed from monosaccharides by reduction of the carbonyl function, which have a sweet taste and are therefore known as sugar substitutes. Examples include: xylitol, sorbitol, mannitol, threitol, and erythritol. Sorbitol and mannitol are particularly preferred.

It is also possible to use flavorings. Examples include vanilla, honey flavoring, apple flavoring, and contramarum, while the skilled man will be familiar with numerous other flavorings which may be used.

Other excipients are flow agents or flow regulators and lubricants. Within the scope of the present invention, silicon dioxide, talc, stearic acid, sodium stearylfumarate, magnesium stearate, and glyceroltribehenate may be used, for example. Preferably, according to the invention, magnesium stearate is used.

The granules according to the invention may contain, in addition to the ingredients mentioned above, disintegrants, sometimes also known as breakdown agents, such as for example modified or pregelatinized starch, CL-PVP (crosslinked PVP).

Preferred fillers according to the invention are glucose, lactose, and starch.

Moreover, the fast dissolving granules according to the invention may contain one or more synthetic or natural pharmaceutically acceptable colorings, preferably indigo carmine.

Naturally, in addition to the excipients mentioned above, other excipients known to the skilled man may be present in the granules according to the invention.

By a suitable choice of ingredients in the granulating liquid and carrier and optionally by adding excipients it is possible to mask the bitter flavor of bromhexin or bromhexinhydrochloride and to produce particularly pleasant tasting granules.

The invention also relates to a process for producing the fast dissolving granules described above, comprising the steps of:
(a) preparing an aqueous granulating liquid containing at least one binder;
(b) spraying the granulating liquid obtained in step (a) on to a carrier containing at least one carbohydrate, in a fluidized bed by the counter-flow method at a constant air supply temperature,
   bromhexin/bromhexinhydrochloride and citric acid being used independently of one another in the granulating liquid and/or carrier.

According to step (a) of the process according to the invention, first of all an aquous granulating liquid is produced which contains one or more of the binders described above in dissolved form, the word “dissolved” being used as defined above for the purposes of the invention. The granulating liquid is therefore preferably obtained as a genuine solution but could also be a dispersion.

Preferably a mixture of two, three, four, five, or more binders is used, while most preferably two or three binders are used. One or more of the excipients described more fully above and optionally one or more carbohydrates such as starch may optionally be added. Furthermore, citric acid and the active substance in the form of bromhexin or bromhexinhydrochloride may also be present in the granulating liquid.

Then, in step (b) of the process according to the invention, the granulating liquid is sprayed on to a carrier at a constant air supply temperature in a fluidized bed by the counter-flow method. The carrier contains at least one carbohydrate, preferably a mixture of two, three, four, five, or more carbohydrates. Most preferably, two or three carbohydrates are used. Excipients, citric acid and the active substance in the form of bromhexin or bromhexinhydrochloride may optionally also be added.

As already explained, the bromhexin(-hydrochloride) active substance is present in either the granulating liquid or the carrier; it may also be present in the carrier and the granulating liquid at the same time. Citric acid is also present either in the granulating liquid or in the carrier and may also be present in the carrier and granulating liquid at the same time. Citric acid and bromhexin or bromhexinhydrochloride are particularly preferably both present in the carrier.

Apart from fluidized bed granulation as used in step (b) of the invention, other granulating methods may also be used, e.g., in a forced mixer, V-Blender, or the one pot process, but these methods are less preferred.

Thus, in step (b) of the process according to the invention, granulation is preferably carried out in a fluidized bed, the fluidized bed chamber used normally being round, while the apparatus may be cylindrical, i.e., it may have a constant diameter over its height. Preferred fluidized bed chambers are those wherein the fluidizing zone is designed be conical and widening out upwards and only the adjacent calming zone is cylindrical after a conical transition member. The process may be carried out batch-wise or continuously, irrespective of the form of the fluidized bed apparatus, but the continuous method is preferred according to the present invention.

In order to produce the fast dissolving granules, according to step (b) of the process according to the invention the granulating liquid obtained in step (a) is preferably introduced into the fluidized bed through a single- or multi-substance nozzle or through a plurality of nozzles. It is preferable according to the invention to use a two-substance nozzle and the substance is atomized for example using compressed air. The arrangement of the nozzle or nozzles and the direction of spraying may be chosen at will provided that the liquid components are distributed substantially uniformly throughout the fluidized bed.

The carrier is placed in the granulating apparatus and fluidized by means of a suitable supply of air from below the fluidized bed. In the meantime the granulating liquid is sprayed on to the carrier from above. The operation is carried out by the counter-flow method.

According to the novel process, the air supply temperature is set to be constant and is preferably in the range from approximately 40° C. to approximately 80° C., particularly approximately 50 to approximately 70° C., most preferably in the region of approximately 60 to approximately 70° C.

The fluidized bed apparatus preferably used has base-plates measuring at least 0.15 m. Particularly preferred fluidized bed apparatus are those with a base-plate having a diameter of 0.4 m to 5 m for example 1.2 m or 2.5 m. However, fluidized bed apparatus with a base-plate having a diameter in excess of 5 m is also suitable. The base-plate used may be a perforated plate, a Conidur plate, a wire mesh or a combination based on a perforated plate with a lattice mesh.

Preferably, in the process according to the invention, spraying speeds of approximately 1 to approximately 20 rpm and particularly approximately 10 to approximately 15 rpm are used, corresponding to approximately 1 to approximately 10 g/min, preferably approximately 3 to approximately 6 g/min.

The granules may be discharged from the fluidized bed through means for grading the granules by size, in a preferred embodiment. This grading may for example be carried out using a screening device or an opposing air current (sifting air) which is regulated so that only particles above a certain size are eliminated from the fluidized bed and smaller particles are retained in the fluidized bed.

Thus, the water contained in the granulating liquid is eliminated. The water content of the products can be adjusted virtually at will but according to the invention the water is almost totally eliminated. Drying is preferably carried out at the same time as the granulation in the fluidized bed. In the fluidized bed the water evaporates off, thereby presumably producing partly dry to fully dried particles from the ingredients of the carrier and granulating liquid, these particles forming the actual granules as the fluidized bed process continues and/or the particles are coated, and the granules are simultaneously dried.

According to another alternative embodiment of the process according to the invention, the granules may be coated during or after the spraying in of the granulating liquid, depending on the choice of binder or binders. The choice of binder or binders determines whether the binder will tend to accumulate around the outside of the granules, e.g., in the form of a coating, or inside the granules. Naturally, a separate process may also be added on in which the granules obtained are additionally coated, e.g., with one or more of the binders and optionally excipients described previously.

A selection of the components and compositions preferably used in the granules according to the invention is listed in Table 1 that follows; neither the components nor the quantities should be regarded as being restrictive, but serve only as a guidance for preparing an optimum composition for every application:

TABLE 1
Selection of components and quantities
preferred according to the invention

| | Formulation | |
|---|---|---|
| Active substance/Excipient | [g/100 g] | Nature/Function(s) |
| Bromhexin-/ | 1 | Active substance |
| bromhexinhydrochloride | | |
| Citric acid | 50 | Salt forming agent |
| PVP 25000 | 2-10 | Binder, coating agent |
| HPMC¹⁾ | 3-10 | Binder, coating agent |
| Makrogol ® 6000²⁾ | 0.1-1 | Coating agent |
| Lactose | ad 100 g | Carrier |
| Glucose | ad 100 g | Carrier |
| Saccharose | ad 100 g | Carrier |
| Mannitol | ad 100 g | Sugar alcohol |
| Sorbitol | ad 100 g | Sugar alcohol |
| Xylitol | ad 100 g | Sugar alcohol |
| Water | q.s. | Solvent |
| Honey flavouring | 0.05-0.2 | Flavoring |
| Vanilla | 0.05-0.2 | Flavoring |
| Apple flavouring | 0.01-0.05 | Flavoring |
| Contramarum | 0.02-0.1 | Flavoring |
| Sunett ® | 0.1-0.5 | Sweetener |
| Na-Saccharine | 0.005-0.05 | Sweetener |

¹⁾Hydroxypropylmethylcellulose
²⁾A polyethyleneglycol

The invention further relates to the use of the fast dissolving granules according to the invention for producing a pharmaceutical composition for the treatment or prevention of illnesses in which bromhexin or bromhexinhydrochloride can be used in a therapeutic or preventive capacity. The water soluble bromhexin/bromhexinhydrochloride granules according to the invention may be administered orally to humans and animals.

The granules according to the invention may also be used as drugs in animals, particularly pigs. Sick animals can be treated through their drinking water by adding the drugs to their drink. Moreover, the granules according to the invention may be administered together with the animal fodder, for example by placing over the fodder (Top dressing) or by compressing or pelletizing it with the fodder.

The advantages of the present invention are various:

For the first time the teaching of the invention provides a water soluble granulated product which contains bromhexin or bromhexinhydrochloride as active substance. The granules according to the invention are water soluble in spite of the fact that bromhexin/bromhexinhydrochloride per se is very difficult to dissolve in water, i.e., the granules dissolve completely in water within a few minutes and release all the active substance. This is defined as “fast dissolving” within the scope of the teaching according to the invention.

Another advantage is the gentle method of manufacturing the bromhexin/bromhexinhydrochloride granules. Because of the formulation and the method of production the granules have very good flow qualities, a constant adjustable content, a narrow range of particle size distribution and low dust formation, ensuring a high quality granulated product. In addition, the granules according to the invention are inexpensive to produce.

Compared with the known Bisolvon® powder (powder containing bromhexinhydrochloride which is known in the art) the granules according to the invention have other advantages: the fact that the granules dissolve quickly makes them substantially easier to take, which may be important not only in patients with difficulty swallowing but also when the granules are administered to animals. The granules according to the invention can be administered in the drinking water, so that, in contrast to the powder known at present, the necessary high concentrations of active substance are achieved for treating or preventing the illnesses in question. The improved flow properties mentioned also allow greater accuracy of dosing. By a suitable choice of the composition of the granules, by the use of suitable binders in the granulating liquid, corresponding carbohydrates in the carrier such as sugars or excipients such as sugar alcohols or sweeteners, it is possible to mask the bitter flavor of bromhexins/bromhexinhydrochloride. If desired, additional coating of the granules may also be carried out. In this way, pleasant tasting granules are obtained having the desired properties.

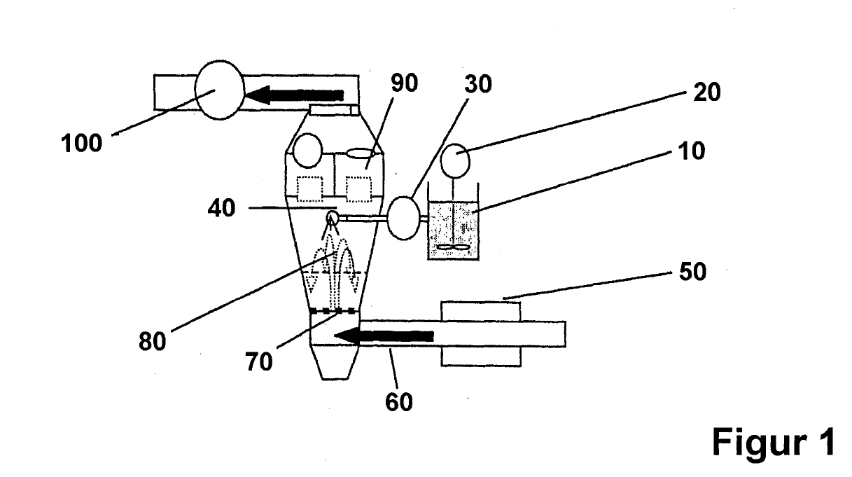

The accompanying FIG. 1 illustrates the teaching of the method according to the invention without restricting the invention thereto. Specifically:

FIG. 1 shows an embodiment of the process according to the invention.

The process according to the invention will be described in detail with reference to the Top spray fluidised bed apparatus manufactured by Glatt of Binzen, shown in FIG. 1.

First of all the granulating liquid is prepared in a container 10 fitted with a stirrer 20. One or more binders, e.g., a mixture of polyvinylpyrrolidone, Eudragit®, and hydroxypropylmethylcellulose are dissolved therein with the addition of bromhexin/bromhexinhydrochloride, in water, with stirring. “Dissolved” in this context also includes dispersed. However, preferably, a granulating solution is produced. A carbohydrate may also be added thereto, as in the present instance, where maize starch is additionally put in. Using a tube pump 30 the granulating liquid thus produced is then conveyed to the two substance nozzles 40 shown which spray the granulating liquid downwards, for example after it has been atomized using compressed air.

The air supply 60 is brought to a constant temperature in a heat store 50, e.g., a temperature in the range from 60° C. to 70° C., preferably 70° C., and is conveyed from below through a screening device 70 to the bed of powder 80 which comprises the carrier which is fluidized by means of the temperature controlled incoming air 60. The carrier is made up of at least one carbohydrate, e.g., a mixture of glucose, lactose, and maize starch. In the present case, citric acid is present in the carrier.

The granulating liquid is thus sprayed on to the fluidized carrier from above, i.e., by a counter-flow method, as a result of which the granules are formed.

Above the two substance nozzle 40, as shown in FIG. 1, there are filters 90 through which the incoming air is first filtered to remove any solid particles carried therein and is then discharged through an exhaust air ventilator 100.

The example that follows serves to illustrate the granules according to the invention. It is to be understood as nothing more than an example of a method, without restricting the invention to its contents.

### EXAMPLE

1. Composition

Granules were produced by the process according to the invention. For this, the compounds specified in Table 2 below were weighed out:

TABLE 2
Compositions used

| Compound | g/100 g | g/preparation |
|---|---|---|
| Bromhexinhydrochloride | 1.0 | 10.0 |
| Anhydrous citric acid | 50.0 | 500.0 |
| Pharmacoat 606* | 2.4 | 24.0 |
| Anhydrous glucose | 46.6 | 466.0 |
| Total | 100.00 | 1000.00 |

*a hydroxypropylmethylcellulose

2. Preparation of the Granulating Solution

201.6 g of water were placed in a product container. Then Pharmacoat 606 was weighed in as the binder, in a 1.2 fold excess. The binder was dissolved in water with stirring.

3. Preparation of the Carrier

The other ingredients specified in Table 2 were pre-screened through a 1 mm screen and weighed together.

4. Preparation of the Granules

The granules were produced using the Top Spray fluidized bed method of Glatt of Binzen. First the apparatus was preheated for 2 minutes (without product). Then the granulating solution was sprayed in through a nozzle onto the carrier in a fluidized bed in a counter-flow. The process conditions and the time taken for the fluidized bed process are shown in Tables 3 and 4 below:

TABLE 3
Process parameters in the fluidized bed process

| | | |
|---|---|---|
| Nozzle: | 1.2 mm | single nozzle, 1 mm |
| | | outside the air head |
| Spray air: | 2 bar | during the warm up phase |
| | | 0.5 bar |
| Dry mixing time: | 2 min | |
| Spraying time: | 42 min (minimum 4 min | |
| | flushing with H₂O) | |
| Drying time: | 2 min | To a product temperature |
| | | of 47° C. |
| Spray medium: | HPMC*/Water 8% | Ambient temperature, 192 g |
| Second spraying: | Water | Ambient temperature, 25 g |

*hydroxypropylmethylcellulose

TABLE 4
Time sequence of the fluidized bed method

| | | | | | Differential | | | |
|---|---|---|---|---|---|---|---|---|
| Time | | Incoming air | | Product | pressure kPA | | Spraying speed | |
| min | Process | m³/h | ° C. | ° C. | Product | Filter | RPM | g/min |
| 0-2 | Warming up | | 70 | 25-52 | 0 | 0 | 0 | 0 |
| 2-5 | Mixing | 5 | 70 | 52-30 | 0.3 | 0 | 0 | 0 |
| From 5 | Wetting | 5 | 70 | 42 | 0.3 | 0.2 | 10 | 3.8 |
| 10 | | 5 | 70 | 42 | 0.3 | 0.4 | 10 | 3.8 |
| 20 | | 5 | 70 | 38 | 0.3 | 0.5 | 10 | 3.8 |
| 30 | | 5 | 70 | 43 | 0.3 | 0.5 | 15 | 6 |
| 40 | | 5 | 70 | 44 | 0.3 | 0.7 | 15 | 6 |
| 43 | Flushing | 5-10 | 70 | 43 | 0.3 | 1.0 | 15 | 6 |
| 47 | Drying | 10-5 | 70-60 | 43 | 0.3 | 1.2 | 0 | 0 |
| 49 | End | | | 47 | 0.3 | 0.9 | 0 | 0 |

1. A fast dissolving granule comprising:
bromhexin or bromhexin hydrochloride,
citric acid,
at least one binder, and
at least one carbohydrate,
wherein the granule is completely dissolvable in a time of less than 15 minutes in a USP Paddle Apparatus at 50 rpm in water at ambient temperature.

2. The fast dissolving granule of claim 1, wherein the bromhexin or bromhexin hydrochloride is present in an amount of approximately 0.5% (w/w) to approximately 2% (w/w).

3. The fast dissolving granule of claim 1, wherein the citric acid is present in an amount of approximately 25% (w/w) to approximately 95.5% (w/w).

4. The fast dissolving granule of claim 1, wherein the at least one binder is present in an amount of approximately 1% (w/w) to approximately 6% (w/w).

5. The fast dissolving granule of claim 1, wherein the at least one carbohydrate is present in an amount of approximately 35% (w/w) to approximately 75% (w/w).

6. A method for using the fast dissolving granule of claim 1 for the therapeutic administration of bromhexin or bromhexin hydrochloride comprising: dissolving the fast dissolving granule of claim 1 and administering the dissolved granule to a human or an animal.

7. A method for using the fast dissolving granule of claim 1 for the therapeutic administration of bromhexin or bromhexin hydrochloride comprising: orally administering the fast dissolving granule of claim 1 to a human or an animal.

8. A method of producing fast dissolving granules comprising preparing an aqueous granulating liquid comprising at least one binder;
preparing a carrier comprising at least one carbohydrate; and
fluidizing the carrier; and
spraying the granulating liquid onto the fluidized carrier to form granules,
wherein bromhexin/bromhexinhydrochloride is present in the aqueous granulating liquid, or the carrier, or both the aqueous granulating liquid and the carrier, and wherein citric acid is present in the aqueous granulating liquid, or the carrier, or both the aqueous granulating liquid and the carrier independent of the bromhexin/bromhexinhydrochloride.

9. The method of claim 8, wherein the bromhexin or bromhexin hydrochloride is present in an amount of approximately 0.5% (w/w) to approximately 2% (w/w).

10. The method of claim 8, wherein the citric acid is present in an amount of approximately 25% (w/w) to approximately 95.5% (w/w).

11. The method of claim 8, wherein the at least one binder is present in an amount of approximately 1% (w/w) to approximately 6% (w/w).

12. The method of claim 8, wherein the at least one carbohydrate is present in an amount of approximately 35% (w/w) to approximately 75% (w/w).

13. The method of claim 8, wherein the bromhexin or bromhexin hydrochloride and the citric acid are present in the carrier.

14. The method of claim 8, wherein the carrier is fluidized by a suitable supply of air from below, and wherein the granulating liquid is sprayed onto the carrier from above, whereby the method is a counter-flow method.

15. The method of claim 8, wherein the carrier is fluidized by a suitable supply of air having a substantially constant temperature.

16. The method of claim 15, wherein the substantially constant temperature is in the range from approximately 40° C. to approximately 80° C.

17. The method of claim 8, wherein the granulating liquid is sprayed onto the carrier at a spraying speed of approximately 1 to approximately 10 g/min.

18. The method of claim 8, further comprising at least partially drying the granules as the granules are being formed.

19. The method of claim 8, wherein the granules are adapted to completely dissolvable in a time of less than 15 minutes in a USP Paddle Apparatus at 50 rpm in water at ambient temperature.

20. The method claim 8, wherein the granulating liquid is prepared in the form of a solution.
